(19) United States
(12) Patent Application Publication
(10) Pub. No.: US 2024/0026470 A1
(43) Pub. Date: Jan. 25, 2024

(54) SYSTEM AND METHOD FOR CHARACTERIZATION AND LABELING OF PLANT VARIETIES

(71) Applicant: Angel Vicente Fernandez Marti, Roseville, CA (US)

(72) Inventor: Angel Vicente Fernandez Marti, Roseville, CA (US)

(21) Appl. No.: 18/471,818

(22) Filed: Sep. 21, 2023

Related U.S. Application Data
(60) Provisional application No. 63/391,086, filed on Jul. 21, 2022.

(51) Int. Cl. C12Q 1/6895; G16B 20/20; C12Q 1/6858

(52) U.S. Cl. CPC C12Q 1/6895; G16B 20/20; C12Q 1/6858; C12Q 2600/156; C12Q 2600/13

(57) ABSTRACT

A system and method for characterizing and identifying a plant variety based on genetic information. Plant samples are subjected to genotyping to obtain genetic information that includes a set of SNP markers. Each of the SNP markers is color-coded according to predefined rules to obtain respective color-coded blocks. The color-coded blocks are arranged by the system to form a matrix, referred to as a molecular ID. This molecular ID is used to characterize plant variety. A QR code associated with the respective molecular ID is attached to respective plant variety.

### CROSS-REFERENCE TO RELATED APPLICATIONS

This application claims priority from of a U.S. Provisional Patent Application Ser. No. 63/391,086, filed on Jul. 21, 1922, the disclosures of which is incorporated herein by reference in their entirety.

### FIELD OF INVENTION

The present invention relates to a system and method for the characterization, identification, labeling, and validation of plant varieties, and more particularly, the present invention relates to a system and method for the characterization, identification, labeling, and validation of plant varieties based on genetic information.

### BACKGROUND

Plant varieties are chiefly described by their morphological characteristics and physical appearance, such as structure, shape, size, odor, and color. Identifying a plant variety by morphological means is the main method of knowing, registering, and describing a new variety. However, such methods of identification have several drawbacks and limitations. Chiefly such method for identification includes subjectivity in the data collected and environmental changes. Many of the morphological traits used to identify differences in domesticated crops are subject to environmental changes. In many pants varieties, such as cannabis plants, many varieties or cultivated varieties appear morphologically similar. Also, many variations in the plant varieties can mislead conclusions about the taxonomic and genetic variability and description of the plants. In the case of cannabis plants, cultivators in same the plant species overall appear the same, and morphological identification is extremely difficult and even sometimes impossible.

DNA molecular markers have been shown to be powerful tools in classifying genetic material and elucidating genetic relationships in plants. Unlike morphological classification, DNA molecular markers are not subject to environmental changes, so no matter where a plant is growing, the DNA profile will remain the same, and genetic classification will be possible. Thus, DNA markers should be the official means to protect breeders' rights and license new cultivars.

The use of DNA markers for plant identification was proposed in 2003 by Paul Hebert to successfully identify any living organism's genetic profiles. Hebert coined this type of identification as “DNA barcoding”. In a similar manner that a barcode can identify a product at a supermarket, short DNA sequences within a plant's genome could also provide identification. This group of scientists discovered that all flowering plants have two genes in common i.e., MatK and RbcL, and they proposed that by sequencing or PCR-amplifying those two genes in different plant taxa, it would be enough to classify plants from different species. Although this is a major advance in plant science and plant taxonomy, those molecular markers are not enough to distinguish between two cultivars/varieties of the same species since the DNA sequence for those two genes will be the same in both cultivars of the same plant species.

Many other studies have been performed with different DNA molecular markers to evaluate the diversity in plants. Within the different molecular markers that have been previously developed, Single Nucleotide Sequence (SNP) markers have been favored over other markers to establish unique genetic identities and assess plants' genetic diversity. These molecular markers are DNA segments and are the preferred technique within crop species due to their abundance, codominance, and high polymorphism with PCR conditions. SNP markers are typically represented by alphabetical symbols such as AA, BB, or AB, since an individual inherits one allele copy from each parent, so the individual genotype at an SNP site is one of those three combinations. When the sequencer does not detect one SNP in one plant but does it in another, the output for that specific SNP appears as NC (not call). One additional advantage of this kind of marker is the high level of reproducibility since they use standardized protocols, so data can be compared between labs.

Every single plant has thousands or even millions of SNPs, depending on their genome size. The abundance of SNPs across plant genomes has made them highly desirable for marker development and plant authentication. High-throughput SNP screening can be achieved effectively by high-density SNP arrays. Developing high-quality SNP loci for array design requires whole genome sequence (WGS) data to assess polymorphism. While this method produces extensive data, it requires complex bioinformatics analysis to filter and select the best few thousand SNPs.

Some studies demonstrated that genotyping plants with low/medium SNP arrays (1000-5000 SNPs) are more than enough to accurately distinguish among cultivars of the same plant species. However, the complexity resides in the analysis and storage of those data. The output results can appear in four categories and are represented by alphabetical symbols: AA, BB, AB, and NC (“not call”). It is not practical to identify a plant species using such complex data. For example, if cannabis breeders or nurseries want to include in each plant, a molecular ID, or a DNA certificate, proving the authenticity of their cultivar, as of now, there is no system to prove this unless they attach a physical document with the thousands of SNPs or molecular markers generated after analysis in the laboratory, which is not practical.

Thus, a need is appreciated for a system and method for taxonomical identification and labeling of a plant variety based on genetic information.

### SUMMARY OF THE INVENTION

The following presents a simplified summary of one or more embodiments of the present invention to provide a basic understanding of such embodiments. This summary is not an extensive overview of all contemplated embodiments and is intended to neither identify critical elements of all embodiments nor delineate the scope of any or all embodiments. Its sole purpose is to present some concepts of one or more embodiments in a simplified form as a prelude to the more detailed description that is presented later.

The principal object of the present invention is therefore directed to a system and method for taxonomical identification and labeling of a plant variety based on low/medium SNP arrays.

It is another object of the present invention to generate a unique, discriminative, and unforgeable genetic identification system that is more accurate than morphological traits, not subject to environmental factors, and very visual.

It is still another object of the present invention that the labels and thus plant varieties can be easily compared.

It is yet another object of the present invention that the method is economical to implement and call up.

It is a further object of the present invention that the system and method allow for authentication and traceability of plant variety.

It is still a further object of the present invention that the genetic information associated with a plant variety is readily available which may have important implications for breeding new cultivars, paternity tests, preservation and distribution of the plant material, marketing, authentication, conservation, and IP of germplasm.

In one aspect, disclosed are a system and method for the implementation of molecular markers in a label. The label includes a graphical representation of information, such as barcodes and QR codes.

In one aspect, disclosed is a method of creating an unforgeable cannabis plant identification and protection stamp/molecular ID based on SNP markers.

In one aspect, disclosed is a method for characterizing and identifying a plant variety, the method comprising subjecting a first plant sample to genotyping to obtain genetic information, the genetic information comprises a set of SNP markers in a defined sequence; color coding each SNP marker of the set of SNP markers based on predefined rules to obtain respective color-coded blocks; arranging the color-coded block according to the defined sequence in a matrix to form a first molecular ID. The method further comprises generating a QR code for the first molecular ID. The set of SNP markers comprises AA, BB, AB, and NC.

In one aspect, the AA is first color-coded, BB is second color-coded, AB is third color-coded, and NC is fourth color-coded, wherein the first color, the second color, the third color, and the fourth color are different. The color-coded blocks are square.

In one aspect, the method further comprises subjecting a second plant sample to the genotyping to form a second molecular ID and comparing the second molecular ID with the first molecular ID to obtain a similarity percentage. The method further comprises overlaying the first molecular ID and the second molecular ID to form an overlaid 2D matrix, wherein differences in the first molecular ID and the second molecular ID are highlighted in the overlaid 2D matrix.

In one aspect, disclosed is a system for characterizing and identifying a plant variety, the system comprises a processor and a memory, and the system is configured to implement a method comprising receiving genetic information of a first plant sample from a sequencer, wherein the genetic information is obtained by genotyping the first plant sample, the genetic information comprises a set of SNP markers in a defined sequence; color coding each SNP marker of the set of SNP markers based on predefined rules to obtain respective color-coded blocks; and arranging the color-coded block according to the defined sequence in a matrix to form a first molecular ID.

### BRIEF DESCRIPTION OF THE DRAWINGS

The accompanying figures, which are incorporated herein, form part of the specification and illustrate embodiments of the present invention. Together with the description, the figures further explain the principles of the present invention and enable a person skilled in the relevant arts to make and use the invention.

FIG. 1 is a flowchart illustrating a method for characterization and labeling of a plant variety, according to an exemplary embodiment of the present invention.

FIG. 2 is a flowchart illustrating the method for identification and validation of a plant variety, according to an exemplary embodiment of the present invention.

FIG. 3 illustrates the results of a sample plant variety comparison with reference materials, according to an exemplary embodiment of the present invention.

FIG. 4 is a flowchart illustrating a method for identification and validation of a plant variety using a QR code, according to an exemplary embodiment of the present invention.

FIG. 5 is a block diagram illustrating the architecture of the system, according to an exemplary embodiment of the present invention.

FIG. 6 is a block diagram illustrating the environment of the system, according to an exemplary embodiment of the present invention.

FIG. 7 is a 2D color-coded matrix generated by the system, also referred to herein as molecular ID, according to an exemplary embodiment of the present invention.

### DETAILED DESCRIPTION

Subject matter will now be described more fully hereinafter with reference to the accompanying drawings, which form a part hereof, and which show, by way of illustration, specific exemplary embodiments. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, the reasonably broad scope for claimed or covered subject matter is intended. Among other things, for example, the subject matter may be embodied as methods, devices, components, or systems. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is to describe particular embodiments only and is not intended to be limiting to embodiments of the invention. As used herein, the singular forms “a”, “an”, and “the” are intended to include the plural forms as well, unless the context indicates otherwise. It will be further understood that the terms “comprises”, “comprising,”, “includes” and/or “including” when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely to illustrate the general principles of the invention since the scope of the invention will be best defined by the allowed claims of any resulting patent.

Disclosed are a system and method for the characterization, identification, labeling, and validation of plant varieties, in particular, the cultivated plant varieties, also commonly referred to as “cultivators”. The disclosed system and method allow for the characterization and identification of a plant variety based on genetic information without any doubt. Moreover, the disclosed system and method simplified the otherwise complex genetic analysis matching process for characterizing and identifying plant species. Also, disclosed is a molecular ID, also referred to herein as a “CERTIFICATE OF CULTIVAR IDENTITY—CCI” and a method for generating and using the same. The molecular ID is a unique character set for each specific plant variety that can be used to readily distinguish a plant variety from others. Each plant variety can be labeled using a QR code that links to the respective molecular ID for identification of the plant variety.

The terms “Molecular ID” and “CERTIFICATE OF CULTIVAR IDENTITY—CCI” are interchangeably used hereinafter. The “Molecular ID” allows breeders, nurseries, and cultivators to identify and validate genetics (cultivars and varieties) through DNA Fingerprinting analysis and guaranteeing authenticity and genetic fidelity. The disclosed system and method allow for extracting and processing the genetic information for generating the respective molecular ID for cultivars using a small amount of plant tissue.

The molecular ID can be genetic proof and evidence to identify a cultivated variety, such as those of plant cannabis, proving its uniqueness. The disclosed system and method can be very useful for the cultivators of the cultivated variety providing certification and authentication for the plant varieties. Each molecular ID is associated with a unique serial number and a QR code. Just by referencing the unique identifiers on the CCIs, one can confidently verify the genetic integrity and consistency of their cultivated variety over time.

The disclosed system and method may employ the unique DNA Fingerprint technology to accurately identify a cultivated variety based on almost 3000 molecular markers. The disclosed system and method use genotyping of plant samples with low/medium SNP arrays for the characterization and labeling of a plant variety. Preferably, the disclosed system and method can be used for cannabis plant varieties. In certain implementations, a set of about 2750 SNP may be developed that can resolve and identify the genetic variability in clones, strains, cultivars, and breeding populations with an efficiency of 99.99% (in the case of identical plants). Genotyping can be generated by using a suitable sequencer, wherein the output generated from a sequencer may be a spreadsheet file or similar data format consisting of thousands of letters such as AA, BB, AB, or NC. The disclosed system and method also provide for simplifying such a complex output of sequencer to an easily distinguishable format I.e., molecular If which is a 2D graphical image comprising color-coded blocks arranged in rows and columns forming a matrix that represents a genotyped sequence. The disclosed system and method can be particularly advantageous by converting and translating the DNA markers (AA, BB, AB, or NC) in a graphical representation i.e., color color-coded 2D matrix. The color-coded blocks in the matrix are arranged in the same molecular position as they would be in the raw data obtained from the sequencer machines. The molecular ID, its generation, and its use are the critical features of the present invention for identifying and distinguishing a plant variety.

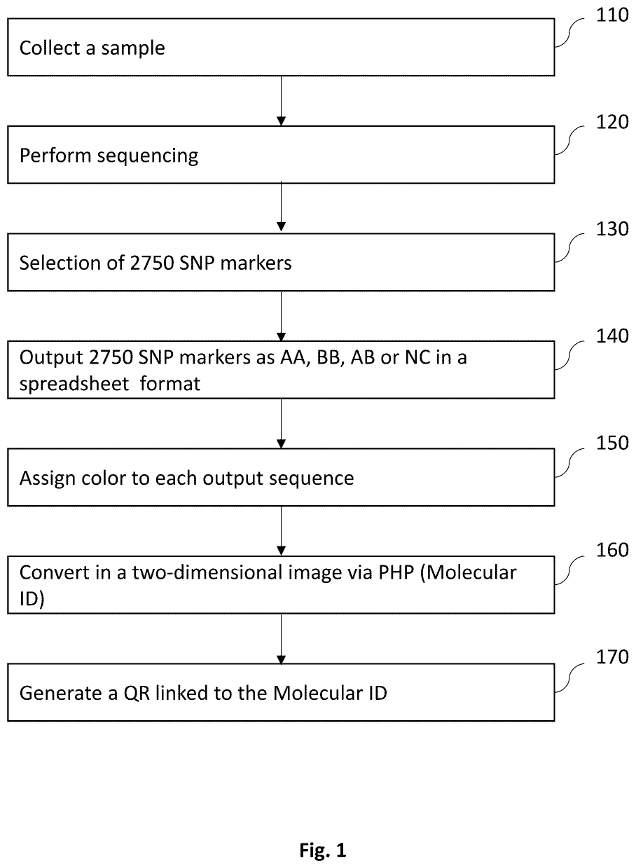

Referring to FIG. 1 illustrates an implementation of the present invention i.e., a method of sequencing and generation of a molecular variety of a plant variety. First, a plant sample suitable for genetic sequencing can be collected (what kind of sample). Typically, a small amount of a sample may be sufficient. The sample can be prepared for genotyping using any of the known methods that may be suitable, at step 110. From the sample, DNA can be extracted using any of the in-house protocols. For example, a phenol/chloroform protocol with specific filtering steps allows the extraction of hundreds of samples with good quality and yield. Genotyping can then be done, at step 120, in which the sample for genotyping can be prepared. Genotyping can be conducted using a suitable sequencer, such as an Illumina chip that includes a set of SNP markers. The selection of printed markers in the Illumina chip can be performed. From the sequencer, raw data can be collected for analysis for a set of markers, for example, the first analysis of the 2750 SNP markers, at step 130. From the raw data, a table with the format of AA, BB, AB, or NC can then be generated, at step 140. Thereafter, the system can convert the double characters to a single-color block, also referred to herein as a color-coded block, at step 150. The locks can be square, rectangular, circular, or any other shape. Each unique pair of SNP markers is assigned a unique color i.e., color-coded. Each tandem of two letters is converted into one unique square. This conversion reduces the total number of characters from 2750 to 1375. SNP marker can be color-coded using a variety of ways, for example, the assignment can be following RGB color codes as follow:
AB=#32466B
NC=#277C43
BB=#917CE8
AA=#4FB071

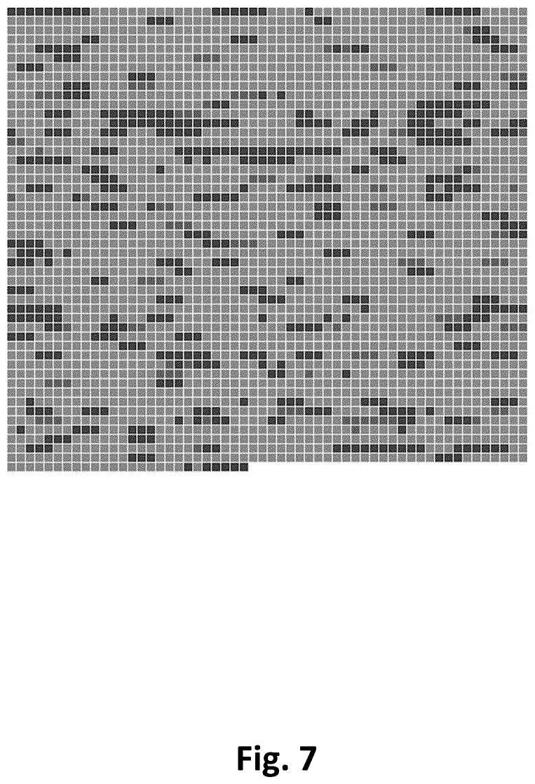

The color-coded data can then be converted into a two-dimensional image of colored coded block, at step 160. FIG. 7 shows an example of a two-dimensional image (shown in greyscale), and this two-dimensional image is referred to herein as the molecule ID. The conversion can be done by using the appropriate algorithm written in a scripting language, such as PHP. This image shown in FIG. 7, may be specific/unique for each cultivated variety and can be used to certify the same. The Molecular ID can be stored in a suitable database and each Molecular ID in the database can have an address. For each molecular ID, a QR code can be generated which embodies the address of the associated molecular ID, at step 170. It is to be understood that the QR code is provided as an example, and any such machine-readable optical image is within the scope of the present invention.

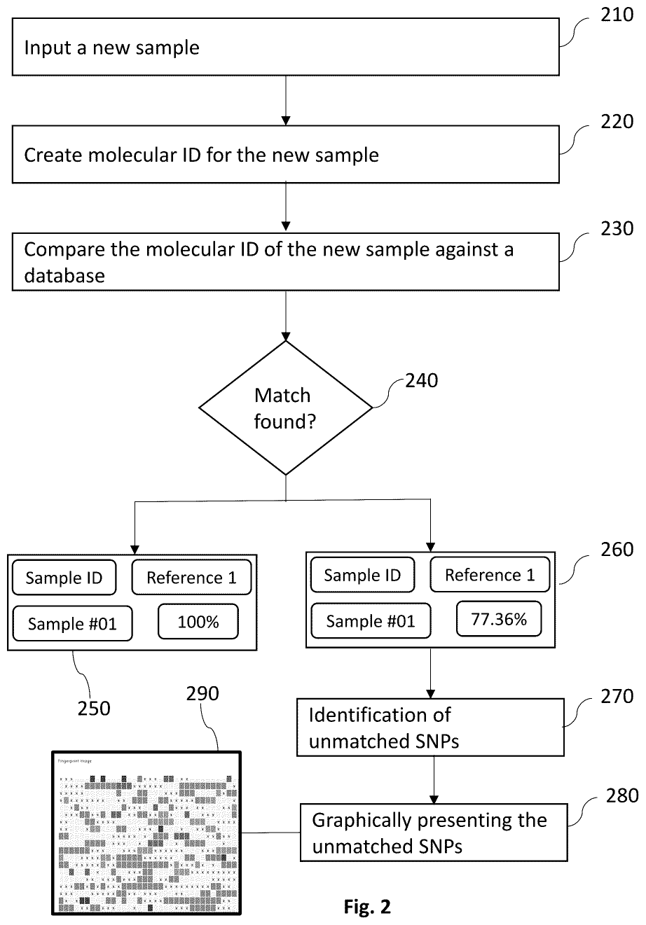

Referring to FIG. 2 which is a flowchart illustrating a method for the identification and validation of a new plant variety. First, a new sample can be taken of an unknown plant variety, at step 210. A new molecular ID can be generated for the unknown plant variety at step 220 using the steps described in FIG. 1. The molecular ID can then be matched by the system with molecular IDs of characterized plant varieties that are stored in a database, at step 240. The system can then present the closest match with the percentage of the match. FIG. 2 shows scenarios 250 and 260. Reference 1 was found to be 100 percent math with the sample ID, and thus the sample ID is identified as Reference 1 in the database. In another scenario, the closest match is reference 1 having a similarity of about 76 percent. The match similarity below a predefined threshold can be considered a different variety. For example, a similarity percentage of more than 95 percent can be considered the same variety, and below 95 percent can be considered a different variety. In case no match can be found in the database, unmatched SNPs with the closest plant a Roth found in the database can be identified, at step 270. The differences can be graphically presented as an overlaid molecular ID matrix in which not-matched blocks can be highlighted. The graphical image 290 can then be displayed by the system.

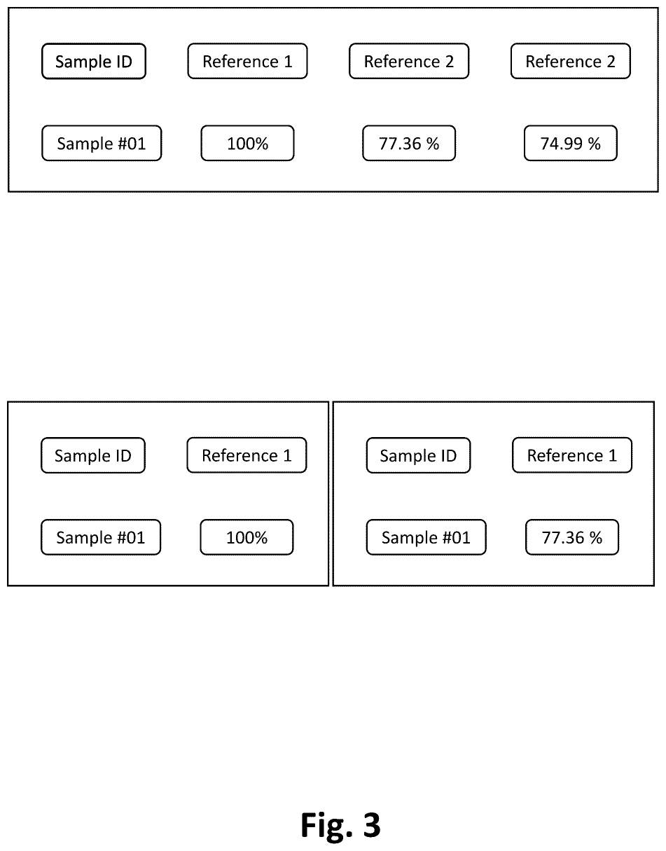

The above describes finding and presenting the closest match reference in a database. However, the system can identify more than one reference in the database. FIG. 3 shows three references with match similarity percentages. Thus, the output can be redefined as desired by the user.

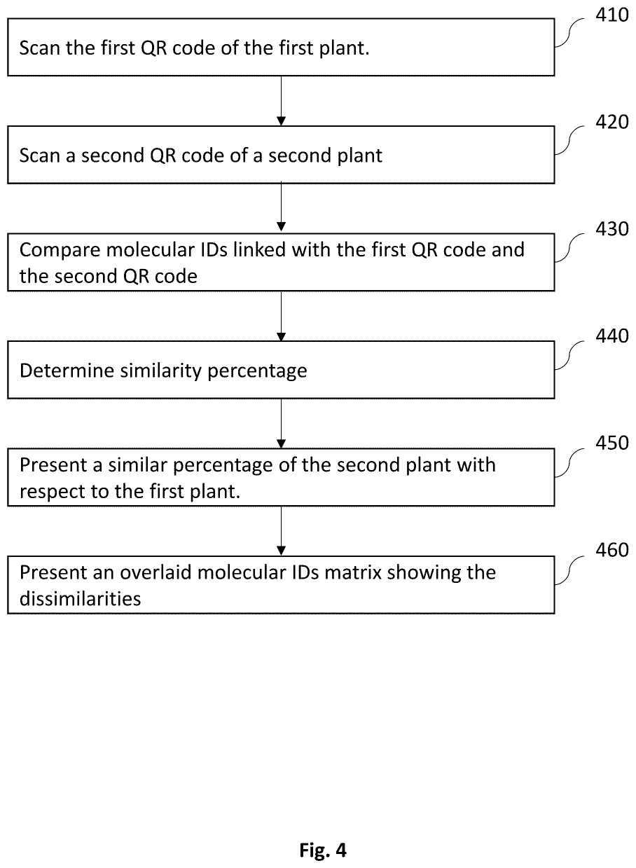

Referring to FIG. 4, which is a flow chart showing another implementation of the invention. First, a user can scan a QR code provided with the plant, at step 410. For example, the QR code can be printed on a label of the plant, or any leaflet attached with plant packaging or pot. Then, the user can scan a QR code of another plant, at step 420. The system can then compare the molecular IDs of the two plant varieties, at step 430. The system can then present the similarity percentage, at step 440. The system can present a similarity percentage of the first plant with the second plant. The system can then present the similarity percentage of the second plant with respect to the first plant, at step 450. The system can then present an overlaid 2D matrix using the respective molecular IDs, wherein the differences can be shown highlighted, at step 460.

In certain implementations, the samples match of more than 99% confidence can be considered genetically identical. In case the samples do not match with more than 99%, another suitable bioinformatic method can be employed to find out the exact percentage similarity between the plant varieties.

In certain implementations, the AA can be converted to a light green square. BB can be transformed into a purple square. AB can be converted to a dark blue square, and the NC can be converted to a dark green square. It is to be understood that the colors, shapes, etc. can be changed. Molecular IDs can be used to identify mismatching molecular markers.

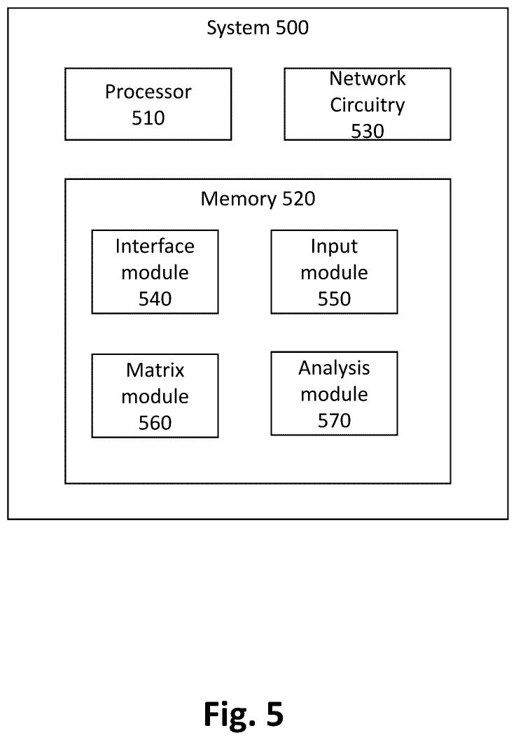

Referring to FIG. 5 which is a block diagram showing an architecture of the disclosed system 500. The system includes a processor 510 and a memory 520. A network circuitry 530 can also be provided for connecting to any external user device or database. The memory can include an interface module 540 that can present an interface for the user to instruct with the disclosed system. An input module 550 may allow for receiving genetic information from a sequencer. The input module may also allow a user to scan a QR code and receive the respective molecular ID for further analysis. The matrix module 560 can generate the molecular ID from the generic information. The analysis module 560 may allow for analyzing the molecular IDs and comparing the same with molecular IDs of other plant varieties to determine similarities.

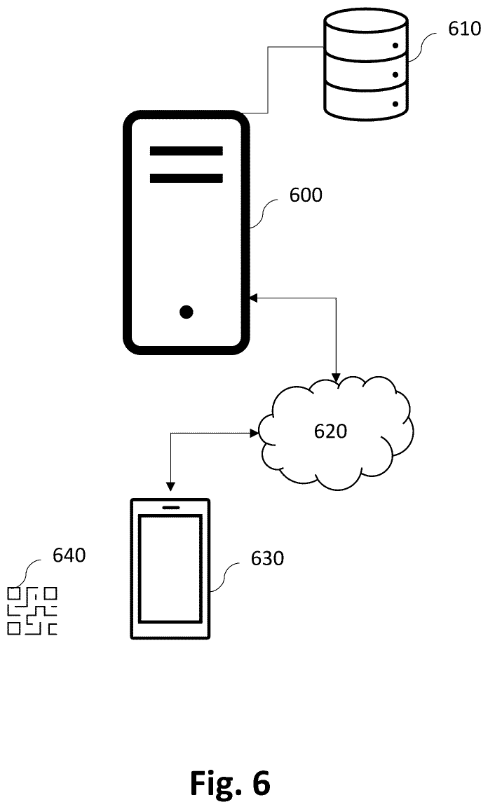

FIG. 6 shows an environmental diagram of the disclosed system 600. The system can connect with database 610 through a network connection. The system can also connect with a user device 630 through a network 620. The user device can be configured to scan a QR code 640. The molecular ID can also be printed as a certificate for certifying a plant variety.

What is claimed is:

1. A method for characterizing and identifying a plant variety, the method comprising:
subjecting a first plant sample to genotyping to obtain genetic information, the genetic information comprises a set of SNP markers in a defined sequence;
color coding each SNP marker of the set of SNP markers based on predefined rules to obtain respective color-coded blocks; and
arranging the color-coded blocks according to the defined sequence in a matrix to form a first molecular ID.

2. The method according to claim 1, wherein the method further comprises:
generating a QR code for the first molecular ID.

3. The method according to claim 1, wherein the set of SNP markers comprises AA, BB, AB, and NC.

4. The method according to claim 3, wherein the AA is first color coded, BB is second color coded, AB is third color coded, and NC is fourth color coded, wherein the first color, the second color, the third color, and the fourth color are different.

5. The method according to claim 1, wherein the color-coded blocks are square.

6. The method according to claim 1, wherein the method further comprises:
subjecting a second plant sample to genotyping to form a second molecular ID; and
comparing the second molecular ID with the first molecular ID to obtain a similarity percentage.

7. The method according to claim 6, wherein the method further comprises:
overlaying the first molecular ID and the second molecular ID to form an overlaid 2D matrix, wherein differences in the first molecular ID and the second molecular ID are highlighted in the overlaid 2D matrix.

8. A system for characterizing and identifying a plant variety, the system comprises a processor and a memory, the system is configured to implement a method comprising:
receiving genetic information of a first plant sample from a sequencer, wherein the genetic information is obtained by genotyping the first plant sample, the genetic information comprises a set of SNP markers in a defined sequence;
color coding each SNP marker of the set of SNP markers based on predefined rules to obtain respective color-coded blocks; and
arranging the color-coded blocks according to the defined sequence in a matrix to form a first molecular ID.

9. The system according to claim 8, wherein the method further comprises:
generating a QR code for the first molecular ID.

10. The system according to claim 8, wherein the set of SNP markers comprises AA, BB, AB, and NC.

11. The system according to claim 10, wherein the AA is first color coded, BB is second color coded, AB is third color coded, and NC is fourth color coded, wherein the first color, the second color, the third color, and the fourth color are different.

12. The system according to claim 8, wherein the color-coded blocks are square.

13. The system according to claim 8, wherein the method further comprises:
subjecting a second plant sample to genotyping to form a second molecular ID; and
comparing the second molecular ID with the first molecular ID to obtain a similarity percentage.

14. The system according to claim 13, wherein the method further comprises:
overlaying the first molecular ID and the second molecular ID to form an overlaid 2D matrix, wherein differences in the first molecular ID and the second molecular ID are highlighted in the overlaid 2D matrix.
